(19) United States
(12) Patent Application Publication
(10) Pub. No.: US 2007/0031778 A1
(43) Pub. Date: Feb. 8, 2007

(54) Care or cleaning device for medical instruments and methods

(75) Inventors: Gerald Helfenbein, Gilgenberg (AT); Daniel Schaffarzick, Elsbethen (AT)

Correspondence Address: KLARQUIST SPARKMAN, LLP, 121 SW SALMON STREET, SUITE 1600, PORTLAND, OR, 97204, US

(73) Assignee: W&H Dentalwerk Burmoos GmbH

(21) Appl. No.: 11/492,374

(22) Filed: Jul. 24, 2006

(30) Foreign Application Priority Data
Aug. 5, 2005 (EP) 05017090.1

(51) Int. Cl. A61C 1/10; A61C 17/02

(52) U.S. Cl. 433/82

(57) ABSTRACT

A care or cleaning device for a medical instrument and a process for determining whether an instrument is connected to one or more coupling devices of the care or cleaning device. Compressed air supplied to each coupling device through a compressed air line causes a backpressure and an increase in the air pressure applied in the compressed air line. If no instrument is connected to a coupling device, then the compressed air can escape through the comparatively large opening in the coupling device and without any great resistance, so there is no increase in the air pressure in the compressed air line. When an instrument is connected, a sensor element senses the air pressure applied in the compressed air line and sends an input signal to a control device. By analyzing the received input signal, the control device detects whether or not the coupling device is occupied by an instrument. In the same way, this process can also be used to differentiate different types of instruments.

### CROSS REFERENCE TO RELATED APPLICATION

The present application claims priority from pending European Patent Application No. 05017090.1, filed Aug. 5, 2005, which is incorporated herein by reference.

### BACKGROUND

1. Field

The present application relates to a care or cleaning device for medical instruments and a process for operating a care or cleaning device.

2. Description of Prior Art

Such a care or cleaning device is known from German Patent Application DE 19 913 962 A1. This device includes multiple couplings for connecting the instruments to be cleaned, whereby a sensor is assigned to each coupling. Each coupling is also connected to a line for cleaning agents with a controllable valve. When an instrument is attached to the coupling, the instrument activates the sensor for delivering a signal that is relayed to a control device. When the control device receives this signal, it opens the valve that is connected by the line to the coupling whose sensor has delivered the signal. When the control device does not receive any signal, it closes the corresponding valve. In this way, only those couplings to which an instrument is connected are treated by the care or cleaning agents.

One disadvantage of this care or cleaning device is that the sensor is activated by the instrument. This means that the sensor must always be arranged in direct proximity to the instrument and thus within the care space. Since water, steam and aggressive cleaning agents and disinfectants are used for cleaning instruments, the sensors are therefore exposed to these media and either must be provided with protective devices so that they do not come in contact with these media or they are subject to increased risk of wear, malfunction or failure. Furthermore, it is impossible to differentiate different types of instruments with this care or cleaning device.

### SUMMARY

Described herein are embodiments of a new care or cleaning device and corresponding methods that address problems of the prior art approaches.

Regardless of the different types and the different designs, many medical instruments have in common the fact that they have an elongated, essentially tubular outer sleeve with only a few openings that are usually small and narrow, leading to the environment. Instruments are understood here to refer in particular to straight and curved handpieces for a wide variety of medical and surgical applications, such as for dental applications, e.g., for drilling cavities, endodontic and prophylactic treatments as well as surgical and orthopedic applications such as sawing or drilling bones. The inside of the outer sleeve is filled with various built-in elements such as motors, shafts, light sources, light guides, media lines, nozzles, valves, tool couplings, control devices, batteries, etc. When such an instrument is connected to the coupling device of a care or cleaning device and compressed air is conveyed through the coupling device, the compressed air must flow through the outer sleeve of the instrument with its few hollow spaces left free by the built-in elements and/or through the media lines with their small diameters, finally emerging into the environment through the small and narrow openings.

An instrument connected to the coupling device and the consequently reduced diameter of the flow path of the compressed air thus results in a backpressure in the compressed air line, connecting the compressed air source to the coupling device, and also results in an increase in the air pressure (flow pressure) applied in the compressed air line. If no instrument is connected to the coupling device, then the compressed air can escape through the comparatively large opening in the coupling device and thus without any great resistance, so that in this case there is little or no backpressure in the compressed air line and thus there is no increase in the air pressure applied in the compressed air line. The inventive process makes use of the increase in pressure of the compressed air when an instrument is connected by having a sensor element activated by the air pressure applied in the compressed air line and sending an input signal to a control device of the care or cleaning device. The control device receives and processes the input signal and, by analysis of the received input signal, detects the occupancy of the coupling device, i.e., whether or not it is occupied by an instrument.

In a first exemplary embodiment, the sensor element is designed as a sensor, and any known sensor may be used, preferably capacitive, inductive or piezoelectric sensors. The input signal generated by the sensor comprises a measured value or is formed by a measured value that represents the air pressure applied in the compressed air line, which corresponds to the elevated flow pressure when the instrument is connected but corresponds to a lower pressure when no instrument is connected. A comparator of the control device compares the received measured value with a predetermined reference value and detects that the coupling device is occupied by an instrument when the measured value exceeds the reference value and/or detects that the coupling device is not occupied when the measured value does not exceed the reference value. Alternatively, by comparison of the measured values, the comparator may detect different types of instruments, in particular a first type of instrument through which the compressed air can pass more easily and/or a second type of instrument that is sealed very well, for example, and thus causes a higher backpressure of the compressed air.

The reference value is stored in a memory and may be an invariable fixed value, which preferably is defined by the manufacturer and is stored in the memory at the time of the manufacture of the care or cleaning device, or alternatively, the reference value may be variable and is preferably modified and saved by the user via an input device, e.g., a potentiometer connected to the control device and the memory. There is thus the option of, for example, recognizing and differentiating several different types of instruments, in particular a first type of instrument that is more permeable to the compressed air than a second type of instrument that is sealed very well. The differentiation of instrument types may be used to advantage to treat the different types of instruments in different ways in a downstream care and/or cleaning process, e.g., using certain care or cleaning steps on only one of the two types of instruments. In particular, a concluding care step in which components of the instrument are provided with a lubricant may be omitted.

A variable reference value may also be determined only at the time of the measurement itself, for example, by using as the reference value one or more measured values of the lower pressure level, i.e., the air pressure in the lines connected to coupling devices to which no instrument is connected, and then all the measured values that differ significantly from this reference value, i.e., by exceeding it, thus represent a coupling device to which an instrument is connected.

In addition, there is the possibility of defining two or more reference values as fixed or variable quantities instead of using one reference value, so that either two or more types of instruments or two or more types of instruments and an unoccupied coupling device can be detected. In addition to memory locations for the reference values, there may also be memory locations for the values of the measured air pressures, so the user can save these values via a control device and actuator elements connected thereto and can assign to them specific care or cleaning processes or process steps, which are stored in the control device. Thus there is a care or cleaning device which is adaptable to any type of instrument which the physician uses. In a following care or cleaning process, the care or cleaning device detects the type of instrument on the basis of the value of the air pressure and selects on the basis of this value the proper care or cleaning process.

In a second exemplary embodiment, the sensor element is designed as a switch, whereby any known switch, micro-switch or integrated electronic pressure switch, preferably a mechanical pressure switch, may be used. The input signal generated by the switch comprises a switching signal or is formed by a switching signal delivered by the switch when the air pressure applied in the compressed air line exceeds the switching point. The switch may be implemented in a known manner as a normally open contact (NO contact) or as a normally closed contact (NC contact), so that according to this approach, the term “switching signal” may also be understood to include the interruption in a continuously applied signal and/or “non-receipt of a signal.”

The control device connected to the switch receives the switching signal, processes it and detects that the coupling device is occupied by an instrument when it receives a first switching signal and/or that the coupling device is not occupied when it receives a second switching signal and/or that the coupling device is occupied by a first type of instrument when it receives a first switching signal and/or that the coupling device is occupied by a second type of instrument when it receives a second switching signal.

It is also possible to provide several switches with different switching points in a compressed air line, each switching point being selected to execute its switching function at a certain air pressure which can be assigned to a certain type of instrument. It is thus also possible to differentiate types of instruments with switches.

In another exemplary embodiment, the control device also sets an occupancy variable for each coupling device and preferably stores it in a memory such that, when the analysis of the received input signal of the sensor element reveals that the coupling device is occupied by an instrument, the occupancy variable assumes a first state, and when analysis of the received input signal reveals that the coupling device is not occupied by an instrument, the occupancy variable assumes a second state. Accordingly, the occupancy variable may assume a first state when analysis of the received input signal reveals that the coupling device is occupied by a first type of instrument and assumes a second state when analysis of the received input signal reveals that the coupling device is occupied by a second type of instrument. If the control device differentiates two or more types of instrument and one unoccupied coupling device or several types of instruments, then the occupancy variables may of course assume a corresponding number of n states.

It is advantageous to set occupancy variables in particular when a care or cleaning device has several coupling devices and the care or cleaning process or at least individual steps thereof take place sequentially, i.e., separately for each instrument or each type of instrument, and only after the end of the process or process step for one instrument or one type of instrument, the process or process step begins for a second instrument or a second type of instrument. The determination of which coupling device an instrument is connected to or not and/or which types of instruments are connected may then be performed advantageously for all coupling devices jointly before the actual care or cleaning process.

After conclusion of the determination of whether an instrument is connected to a coupling device and/or which type of instrument is connected and before the start of the actual care or cleaning process, the control device releases or enables a controllable care or cleaning agent unit of the care or cleaning device and/or parts thereof for the coupling device for which the analysis of the input signal reveals that it is occupied by an instrument. The care or cleaning agent unit is connected by lines to each coupling device, so that in the following care or cleaning process, the instrument connected to the coupling device can be supplied with care or cleaning agents according to an automatic program sequence of the control device. If one coupling device is not occupied by an instrument, the control device does not release the controllable care or cleaning agent unit and/or parts thereof, so that this coupling device is not supplied with care or cleaning agents.

If the coupling device is occupied by a first type of instrument and a second type of instrument, the control device releases a controllable care or cleaning agent unit of the care or cleaning device and/or parts thereof for all coupling devices if both types of instruments are to be cared for with a care or cleaning agent and/or the control device releases only coupling devices with those types of instruments that are to be cared for with a care or cleaning agent that is not provided for the other type of instrument. Accordingly, when the occupancy variable assumes a first, second or n-th state, the control device proceeds so that for each state, the care or cleaning agent unit can be released for a coupling device or not.

The care or cleaning agent unit that can be controlled by the control device preferably comprises one or more controllable blocking devices that can interrupt and cut off the connection between the care or cleaning agent unit and the coupling device. This preferably includes valves, in particular solenoid valves, or slide valves that are closed in a basic state and are opened by the control operation, or pumps that are activated by the control operation and convey media. The same controllable blocking devices are also present in the air lines between the air source and the coupling devices.

Disclosed below are representative embodiments of apparatus and methods. The described structures and methods should not be construed as limiting in any way. Instead, the present disclosure is directed toward all novel and nonobvious features, aspects, and equivalents of the various embodiments, alone and in various combinations and sub-combinations with one another. The disclosed technology is not limited to any specific aspect, feature, or combination thereof, nor do the disclosed materials, structures, and methods require that any one or more specific advantages be present or problems be solved. For the sake of simplicity, the attached figure may not show the various ways in which the disclosed apparatus and methods can be used in conjunction with other systems, methods, and apparatus.

### BRIEF DESCRIPTION OF THE DRAWING

FIG. 1 shows a schematic diagram of the care or cleaning device.

### DETAILED DESCRIPTION

The care or cleaning device 1 comprises a care space 2 and at least one other partial space 3 which are separated from one another by a wall 4. The care space 2 is sealed with respect to the environment so that no care or cleaning media can escape to the outside during a care or cleaning process. The instrument(s) to be cleaned can be introduced into the care space 2 through a closable opening, e.g., equipped with a pivotable or displaceable door. In addition, one or more coupling devices 5A, 5B are provided for connecting the instrument(s) 6 to be cleaned. The instrument 6 shown here is an angled dental handpiece in which several media channels, e.g., for water and compressed air, are arranged, and run from the coupling 7 through the handle sleeve section 8 up to or close to the head 9 of the handpiece.

However, the angled manual dental handpiece shown here is only one possible instrument of a plurality of medical or surgical instruments or instrument parts, e.g., handpiece heads with a neck section that can be connected to a coupling device 5A, 5B.

The coupling device 5A, 5B may essentially be comprised of any known coupling device, e.g., a screw coupling, a plug coupling, a rotary coupling or a quick coupling.

However, it preferably comprises a coupling that resembles a standardized motor connector 10 or a turbine handpiece coupling, so that for the user, the connection of the instrument 6 to be cleaned does not differ from the connection of the instrument 6 to a drive motor. For connecting instruments with a connection pattern that does not fit the coupling device 5A, 5B, it is possible to provide adapters that can be detachably connected to the coupling device 5A, 5B at one end and can be detachably connected to the instrument 6 at the other end. Of course, several different coupling devices or coupling devices with different connection patterns may also be provided in a care or cleaning device 1.

The coupling that resembles a standardized motor connector 10 comprises a stop 11, which is attached in or on the wall 4 of the care or cleaning device 1 and at the same time limits the depth of insertion of the coupling pin 12 into the instrument 6. The coupling pin 12 has the same cylindrical shape and the same dimensions as the standardized coupling pin of a motor and also has the same surface structure. Two boreholes arranged radially in relation to the longitudinal axis of the coupling pin 12 open at the surface of the coupling pin 12, each borehole being surrounded by two gaskets 14 arranged in ring grooves. Cleaning media can be delivered through these boreholes to the instruments 6 that are to be cleaned, whereby a ring channel on the inside of the coupling tube of the instrument 6 is preferably arranged over a borehole when the instrument 6 is attached to the coupling pin 12. One of the ring channels is especially preferably connected to a line carrying water on the inside of the instrument 6 and the second ring channel is connected to a line carrying compressed air on the inside of the instrument 6, so that these media lines can be supplied selectively with cleaning agent(s) and/or care agent(s).

The coupling pin 12 also comprises an elastic catch nose 13 which presses against the inside of the coupling pipe of the instrument 6 when the instrument 6 is attached to the coupling device 5A, 5B and thus additionally secures the instrument 6 on the coupling pin 12.

The partial space 3 of the care or cleaning device 1 comprises the care or cleaning agent unit 15, the control device 16, a connection to a compressed air source 17 and a connection to a current source (not shown).

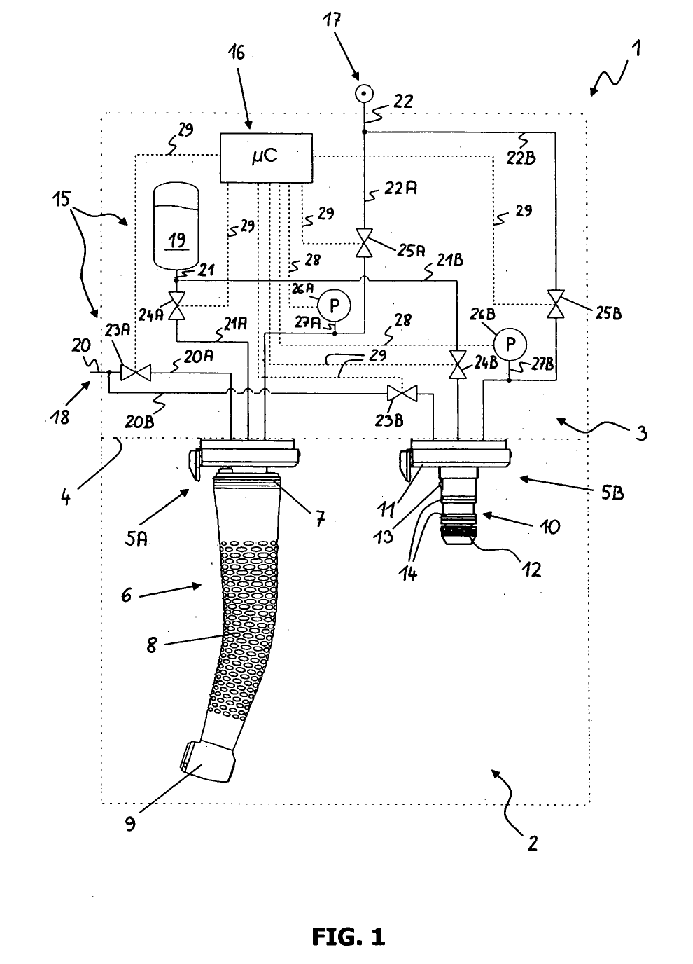

Depending on the type of care or cleaning device 1, the care or cleaning agent unit 15 comprises a connection to a water source 18 and/or to a source for a cleaning agent and/or to a source for a disinfectant and/or to a source for a lubricant. The sources for cleaning agent, disinfectant and lubricant are preferably designed as containers, whereby a container 19 is illustrated as symbolic of these sources in the schematic diagram in FIG. 1. However, the inventive care or cleaning device 1 may comprise one or more containers for one or more cleaning agents, disinfectants and/or lubricants. The connection to a water source 18 is preferably designed as a connection to a water line.

As shown in FIG. 1, each of these sources is connected to each coupling device 5A, 5B by its own separate line. The connection to a water source 18 is via line 20A with coupling device 5A and via a line 20B with coupling device 5B. Container 19 is connected to coupling device 5A via line 21A and to coupling device 5B via line 21B, just as additional containers for additional care or cleaning media may be connected to each coupling device 5A, 5B via separate lines. The lines 20A, 20B and 21A, 21B each branch off from a common line 20, 21.

The connection to a compressed air source 17 is also connected to each coupling device 5A, 5B by its own line 22A, 22B, each of which branches off from a common line 22. The compressed air lines 22A, 22B continue on the inside of the couplings 5A, 5B in a line and/or a borehole, preferably as a central borehole in the coupling pin 12, which opens into the care space 2 through an opening at one end of the coupling pin 12. A pressure regulator may preferably be provided in the line 22, making it possible for the care or cleaning device 1 to be connected to compressed air sources with a different or variable air pressure, but operated with a uniform air pressure. In most cases, the air pressure delivered by the different compressed air sources is in the range of 4 to 10 bar, so that a pressure regulator that lowers the air pressure to a value between 2 and 4 bar is preferably provided.

If the care or cleaning device 1 comprises additional coupling devices, then each coupling device is likewise connected via a separate line to the respective sources 17, 18, 19. It is of course also possible for the lines 20A, 20B, 21A, 21B, 22A, 22B not to branch off from a main line 20, 21, 22 but instead to be connected directly to the respective sources 17, 18, 19.

The care or cleaning agent unit 15 comprises multiple controllable cutoff devices 23A, 23B, 24A, 24B, that are each designed as valves and are arranged in the lines 20A, 20B, 21A, 21B. Cutoff devices 25A, 25B designed as valves, in particular as solenoid valves, are also provided in the air lines 22A, 22B. Cutoff devices 23A, 23B, 24A, 24B, 25A, 25B serve to open or close the respective line 20A, 20B, 21A, 21B, 22A, 22B to control the flow of the respective medium through the line. Each of these cutoff devices 23A, 23B, 24A, 24B, 25A, 25B is connected to the control device 16 by a line 29. The control device 16 may be designed as a mechanical timer, a circuit or a computer, especially preferably as a microcontroller.

Each coupling device 5A, 5B is also assigned a sensor element 26A, 26B, preferably a sensor or switch that is set up in partial space 3. Each sensor element 26A, 26B is connected directly to the compressed air line 22A, 22B and in particular is arranged in the compressed air line 22A, 22B or in a branch 27A, 27B of the compressed air line 22A, 22B. Alternatively, the sensor elements 26A, 26B may be arranged on the circuitboard of the control device 16. Each sensor element 26A, 26B is connected to the control device 16 by a line 28. The sensor elements 26A, 26B can be activated by the air pressure applied or rising in the respective compressed air line 22A, 22B, i.e., they deliver a corresponding input signal to the control device 16 over the line 28.

The functioning of the care or cleaning device 1 in conjunction with a process for determining the occupancy of the coupling devices 5A, 5B is described below, i.e., whether one or more of the coupling devices 5A, 5B is occupied by an instrument 6 and/or by which type of instrument they are occupied. This process takes place as a separate process before the start of the actual care or cleaning process or as a subordinate process of the care or cleaning process, with the goal of supplying care and cleaning agents in the subsequent care or cleaning process only to those coupling devices that are occupied by an instrument 6 and/or when different types of instruments are connected to the coupling devices 5A, 5B to allow these types of instruments to be treated with different care or cleaning processes or process steps.

When the care or cleaning device 1, which is connected to a water source and to a compressed air source and whose sources for cleaning agent and/or disinfectant and/or lubricant are filled, is turned on and/or started, the care or cleaning agent unit 15 and/or all cutoff devices 23A, 23B, 24A, 24B, 25A, 25B are not released, i.e., they are closed or inactive, so that no medium can be conveyed through the corresponding lines 20A, 20B, 21A, 21B, 22A, 22B. The user connects the instrument(s) 6 that are to be cleaned to the coupling devices 5A, 5B and starts the process by depressing a button. Therefore, one or more buttons with switches are provided on the care or cleaning device 1 in a known way, these switches being connected to the control device 16 so that the user can start one or more operating programs for care or cleaning processes stored in a memory in the control device 16, select from several preset operating programs for care or cleaning process or modify various operating parameters of care or cleaning processes.

In a first step, the cutoff devices 25A, 25B of the compressed air lines 22A, 22B are opened so that compressed air from the compressed air source can flow through the connection 17, the compressed air lines 22, 22A, 22B to the coupling device 5A, 5B. The opening of the cutoff devices 25A, 25B may optionally take place individually for each cutoff device 25A, 25B, so that the process takes place sequentially for each coupling device 5A, 5B or for all cutoff devices 25A, 25B simultaneously. With simultaneous control, it is necessary to ensure through suitable means, e.g., throttles, that the different pressure conditions in the compressed air lines 22A, 22B are not equalized or mutually influenced, thereby falsifying the measurements. If an instrument 6 is connected to one of the coupling devices, as shown in FIG. 1 for coupling device 5A, for example, then the compressed air must flow through the instrument 6 to be able to escape to the environment. Because of the narrowed flow path through the instrument 6, there is a backpressure, leading to an increase in the air pressure (flow pressure) in the line 22A. However, the compressed air flowing into line 22B is not influenced by any instrument 6, so that it can escape into the care space 2 without any mentionable resistance or backpressure. For example, the dynamic pressure of the incoming compressed air is approximately 2.5 bar after it has passed through the pressure regulator in line 22 and the flow pressure in line 22A is approximately 2.0 bar due to the connected instrument 6. In line 22B without the connected instrument 6, the flow pressure amounts to approximately 1.5 bar. The reference value with which the two measured pressure values are compared is set at approximately 1.7 bar in this example.

Sensor elements 26A, 26B are activated by the air pressure applied in the respective compressed air lines 22A, 22B and each sends an input signal to the control device 16 over the lines 28. The input signal is processed and analyzed by the control device, depending on whether the sensor element 26A, 26B is a sensor or a switch, in the manner described above, so the description will not be repeated here to avoid repetition.

Different types of instruments that are connected to the coupling devices 5A, 5B are determined in the same way, i.e., the different types of instruments are again detected by the change in air pressure in the respective compressed air lines 22A, 22B and the input signals generated thereby.

After determining which coupling device 5A, 5B an instrument 6 is connected to, the control device 16 controls the cutoff devices of the care and cleaning agent unit 15 and the compressed air lines 22A, 22B which are assigned to a coupling device 5A, 5B to which an instrument 6 is connected and opens and/or activates it, so that compressed air, water as well as the care and cleaning agents are conveyed only to these coupling devices in the following care or cleaning process. Thus, the cutoff devices 23A, 24A, 25A are opened in FIG. 1 and the cutoff devices 23B, 24B, 25B remain closed.

If different types of instruments have been determined by this process, then the control device 16 starts—either automatically or on selection by the user—a care or cleaning process that performs certain process steps only for a certain type of instrument. For example, handpieces operated by compressed air, so-called turbine handpieces, and electrically operated tartar-removing handpieces may be connected to the coupling devices 5A, 5B. On recognition of the types of instruments, the compressed air escapes much less easily through the tartar-removing handpiece, so that the air pressure in the compressed air line that is connected to the tartar-removing handpiece via the coupling device 5A, 5B increases much more greatly than in the compressed air line connected to the turbine handpiece.

In the following care or cleaning process, for example, a concluding care step in which lubricant is conveyed into the instruments is skipped for the tartar-removing handpieces, because these instruments do not have any moving parts that must be lubricated. Thus the cutoff devices in the lines that carry lubricant from a lubricant source to the coupling devices 5A, 5B to which the tartar-removing handpieces are connected are kept closed or inactive by the control device 16, which also releases only the other cutoff devices, i.e., opens or activates them.

Alternatively, with a care and cleaning device having both internal and external cleaning, there is also the possibility of performing only an external cleaning on a first type of instrument and performing both an external cleaning and an internal cleaning on a second type of instrument. Accordingly, with the type of instrument that is cleaned only externally, the only cutoff devices released, i.e., opened or activated by the control device are those in the media lines that connect media sources for the external cleaning to nozzles in the care space.

In view of the many possible embodiments to which the disclosed principles may be applied, it should be recognized that the illustrated embodiments are only preferred examples and should not be taken as limiting in scope. Rather, the scope is defined by the following claims. We therefore claim all that comes within the scope and spirit of these claims.

What is claimed is:

1. A process for determining whether a coupling device of a care or cleaning device for medical instruments is occupied by an instrument and/or by which type of instrument it is occupied, the process comprising:
conveying compressed air through a coupling device via a compressed air line allocated to the coupling device;
providing a sensor element activated by air pressure applied in the compressed air line, wherein in response to sensing air pressure, the sensor element sends an input signal to a control device of the care or cleaning device; and
detecting the occupancy of the coupling device and/or the type of instrument connected to the coupling device with the control device by analyzing the received input signal.

2. The process according to claim 1, wherein the input signal comprises a measured value determined by the sensor element that reflects the air pressure applied in the compressed air line, and detecting the occupancy of the coupling device and/or the type of instrument connected to the coupling device comprises comparing the received measured value with a reference value,
wherein the control device detects at least one of the following conditions:
the coupling device is occupied by an instrument if the measured value exceeds the reference value, or
the coupling device is not occupied if the measured value does not exceed the reference value, or
the coupling device is occupied by a first type of instrument if the measured value exceeds the reference value, or
the coupling device is occupied by a second type of instrument if the measured value does not exceed the reference value.

3. The process according to claim 1, wherein the input signal comprises a switching signal of a switch, and detecting the occupancy of the coupling device and/or the type of instrument connected to the coupling device comprises detecting the switching signal,
wherein the control device detects at least one of the following conditions:
the coupling device is occupied by an instrument when the coupling device receives a first switching signal, or
the coupling device is not occupied when the coupling device receives a second switching signal, or
the coupling device is occupied by a first type of instrument when the coupling device receives a first switching signal, or
the coupling device is occupied by a second type of instrument when the coupling device receives a second switching signal.

4. The process according to claim 1, wherein the control device sets an occupancy variable for each coupling device, and based on analysis of the received input signal by the control device, the occupancy variable is assigned at least a first state or a second state,
wherein the occupancy variable state is assigned according to at least one of the following conditions:
the coupling device is connected to an instrument, or
the coupling device is not connected to an instrument, or
the coupling device is connected to a first type of instrument, or
the coupling device is connected to a second type of instrument.

5. The process according to claim 4, wherein the control device stores the occupancy variable in a memory.

6. The process according to claim 4, wherein the occupancy variable is assigned at least a first state, a second state or a third state.

7. The process according to claim 1, wherein the control device controls a care or cleaning agent unit of the care or cleaning device before the start of a care or cleaning process, and wherein, upon occurrence of a predetermined event as detected by analysis of the input signal, the control device controls the care or cleaning agent unit to release media,
wherein the predetermined event includes at least one of the following:
the coupling device is occupied by an instrument, or
the coupling device is occupied by a first type of instrument.

8. The process according to claim 4, wherein the control device controls a care or cleaning agent unit of the care or cleaning device before the start of a care or cleaning process, and wherein, upon occurrence of a predetermined event as detected by analysis of the input signal, the control device controls the care or cleaning agent unit to release media,
wherein the predetermined event includes at least one of the following:
the coupling device is occupied by an instrument, or
the coupling device is occupied by a first type of instrument, or
the occupancy variable is assigned to a first state, or
the occupancy variable is assigned to a second state.

9. A care or cleaning device for medical instruments comprising:
one or more coupling devices for connecting one or more instruments,
connections to a compressed air source, a care or cleaning agent unit and a control device, whereby each coupling device is assigned a compressed air line connecting the coupling device to the compressed air source and a sensor element, whereby each sensor element is connected to the control device, and delivers an input signal to the control device when activated, so that by analyzing the input signal, the control device detects the occupancy of at least one coupling device and/or the type of instrument connected to the at least one coupling device,
wherein the sensor element is activated by the air pressure applied in the compressed air line.

10. The care or cleaning device according to claim 9, wherein the sensor element comprises a capacitive, inductive or piezoelectric sensor or a switch.

11. The care or cleaning device according to claim 9, wherein the sensor element comprises a mechanical switch.

12. The care or cleaning device according to claim 9, wherein the care or cleaning device comprises a care space and a partial space separate from the care space, and wherein the sensor element is arranged in the partial space.

13. The care or cleaning device according to claim 9, wherein each sensor element is connected directly to the compressed air line.

14. The care or cleaning device according to claim 9, wherein each sensor is arranged in the compressed air line or in a branch line of the compressed air line.

15. The care or cleaning device according to claim 9, wherein the control device comprises a circuit board which carries at least a part of the sensor elements.

16. The care or cleaning device according to claim 9, further comprising a care or cleaning agent unit controllably connected to the control device, wherein when analysis of the input signal reveals that a coupling device is occupied by an instrument, the care or cleaning agent unit can be controlled by the control device so that media is conveyed from the care or cleaning agent unit into said instrument.

17. The care or cleaning device according to claim 9, further comprising a care or cleaning agent unit controllably connected to the control device, wherein when analysis of the input signal reveals that a coupling device is occupied by a specific type of instrument, the care or cleaning agent unit can be controlled by the control device so that media is conveyed from the care or cleaning agent unit into said instrument.
